(19) United States
(12) Patent Application Publication
(10) Pub. No.: US 2013/0317388 A1
(43) Pub. Date: Nov. 28, 2013

(54) ZERO-HEAT-FLUX TEMPERATURE MEASUREMENT DEVICES WITH PERIPHERAL SKIN TEMPERATURE MEASUREMENT

(71) Applicants: Mark T. Bieberich, Lakeway, TX (US); John P. Rock, Minneapolis, MN (US)

(72) Inventors: Mark T. Bieberich, Lakeway, TX (US); John P. Rock, Minneapolis, MN (US)

(73) Assignee: ARIZANT HEALTHCARE INC., St. Paul, MN (US)

(21) Appl. No.: 13/983,350

(22) PCT Filed: Feb. 2, 2012
(86) PCT No.: PCT/US12/00059

§ 371 Date: Aug. 2, 2013

Related U.S. Application Data
(60) Provisional application No. 61/463,393, filed on Feb. 16, 2011.

(51) Int. Cl. A61B 5/01; G01K 17/00; A61B 5/00

(52) U.S. Cl. CPC A61B 5/01; A61B 5/746; G01K 17/00
USPC 600/549; 374/29

(57) ABSTRACT

A zero-heat-flux temperature measurement device has first and second flexible substrate layers sandwiching a layer of thermally insulating material. A heater trace disposed on the first substrate layer defines a heater facing one side of the layer of thermally insulating material and including a central portion surrounding a first thermal sensor and a peripheral portion surrounding the central portion. A second thermal sensor is disposed on the second substrate layer facing an opposing side of the layer of thermally insulating material, and third thermal sensor is disposed on the second substrate layer facing the opposing side of the layer of thermally insulating material. The second and third thermal sensors are separated so as to provide respective skin temperatures at separate locations in a skin surface area where a tissue temperature is to be measured.

### PRIORITY

This application claims priority to U.S. Provisional Application for Pat. No. 61/463,393, filed Feb. 16, 2011.

### RELATED APPLICATIONS

This application contains subject matter related to subject matter of the following US patent applications, all commonly-owned herewith:

U.S. patent application Ser. No. 12/584,108, filed Aug. 31, 2009;

U.S. patent application Ser. No. 12/798,668, filed Apr. 7, 2010; and

U.S. patent application Ser. No. 12/798,670, filed Apr. 7, 2010.

### BACKGROUND

The subject matter relates to a device for use in the estimation of deep tissue temperature (DTT) as an indication of the core body temperature of humans or animals. In particular the subject matter relates to a zero-heat-flux temperature measurement device with provision for measuring temperature at multiple locations in a skin temperature measurement area.

Deep tissue temperature measurement is the measurement of the temperature of organs that occupy cavities of human and animal bodies (core body temperature). DTT measurement is desirable for many reasons. For example, maintenance of core body temperature in a normothermic range during the perioperative cycle has been shown to reduce the incidence of surgical site infection; and so it is beneficial to monitor a patient's body core temperature before, during, and after surgery. Of course noninvasive measurement is highly desirable, for the safety and the comfort of a patient, and for the convenience of the clinician. Thus, it is useful to obtain a noninvasive DTT measurement by way of a device placed on the skin.

Noninvasive measurement of DTT by means of a zero-heat-flux device was described by Fox and Solman in 1971 (Fox R H, Solman A J. A new technique for monitoring the deep body temperature in man from the intact skin surface. J. Physiol. Jan 1971:212(2): pp 8-10). Because the measurement depends on the absence of heat flux through the skin area where measurement takes place, the technique is referred to as a “zero-heat-flux” (ZHF) measurement. The Fox/Solman system, illustrated in FIG. 1, estimates core body temperature using a ZHF temperature measurement device 10 including a pair of thermistors 20 separated by layer 22 of thermal insulation. A difference in the temperatures sensed by the thermistors 20 controls operation of a heater 24 of essentially planar construction that stops or blocks heat flow through a skin surface area contacted by the lower surface 26 of the device 10. A comparator measures the difference in the sensed temperatures and provides the difference measurement to a controller 30. The heater 24 is operated for so long as the difference is non-zero. When the difference between the sensed temperatures reaches zero, the ZHF condition is satisfied, and the heater 24 is switched on and off as needed to maintain the ZHF condition. The thermistor 20 at the lower surface 26 senses the temperature of the skin surface area and its output is amplified at 36 and provided at 38 as the system output. Togawa improved the Fox/Solman technique with a DTT measurement device structure that accounted for multidimensional heat flow in tissue. (Togawa T. Non-Invasive Deep Body Temperature Measurement. In: Rolfe P (ed) Non-Invasive Physiological Measurements. Vol. 1. 1979. Academic Press, London, pp. 261-277). The Togawa device, illustrated in FIG. 2, encloses a Fox and Solman-type ZHF design in a thick aluminum housing 11 with a cylindrical annulus construction that reduces or eliminates radial heat flow from the center to the periphery of the device.

The Fox/Solman and Togawa devices utilize heat flux normal to the body to control the operation of a heater that blocks heat flow from the skin through a thermal resistance in order to achieve a desired ZHF condition. This results in a construction that stacks the heater, thermal resistance, and thermal sensors of a ZHF temperature measurement device, which can result in a substantial vertical profile. The thermal mass added by Togawa's cover improves the stability of the Fox/Solman design and makes the measurement of deep tissue temperature more accurate. In this regard, since the goal is zero heat flux through the device, the more thermal resistance the better. However, the additional thermal resistance adds mass and size, and also increases the time used to reach a stable temperature at start up and impairs the device's ability to timely report rapid changes in temperature.

The size, mass, and cost of the Fox/Solman and Togawa devices do not promote disposability. Consequently, they must be sanitized after use, which exposes them to wear and tear and undetectable damage. The devices must also be stored for reuse. As a result, use of these devices raises the costs associated with zero-heat-flux DTT measurement and can pose a significant risk of cross contamination between patients. It is thus desirable to reduce the size and mass of a zero-heat-flux DTT measurement device, without compromising its performance, in order to promote disposability after a single use.

### SUMMARY

In an aspect of this disclosure, a ZHF temperature measurement device is constituted of a flexible substrate and a ZHF electrical circuit disposed on a surface of the flexible substrate having the capability of measuring a temperature difference between skin surface locations separated in a lateral direction of a surface of the device which contacts a skin surface area wherein the skin surface locations are contained.

In another aspect of this disclosure, a temperature difference is measured across a surface area that is contacted by a surface of the heater of a ZHF temperature measurement device.

In another aspect of this disclosure, a temperature difference is measured between inner and peripheral portions of a skin surface area contacted by a substrate surface of a ZHF temperature measurement device constituted of a flexible substrate and an electrical circuit.

A ZHF temperature measurement device constituted of a flexible substrate supporting an electrical circuit includes a heater and thermal sensors disposed on a surface of the substrate.

In some aspects, the device includes at least three thermal sensors: a first thermal sensor that senses the heater temperature, a second thermal sensor separated in a first direction from the first thermal sensor that senses a skin temperature at a first location within the skin surface area, and a third thermal sensor separated from the second thermal sensor in a second direction that senses a skin temperature at a second location of the skin surface area.

In some other aspects, the first location within the skin surface area is a central location of the skin surface area and the second location is displaced toward the periphery of the skin surface from the central location.

In still other aspects, a zero-heat-flux DTT measurement device is constituted of first and second flexible substrate layers, a heater disposed on a surface of the first substrate layer surrounding an unheated zone of the first substrate layer, a first thermal sensor disposed on the first substrate layer, in the unheated zone, a second thermal sensor disposed on the second substrate layer at a location within a projection of the heater, and a third thermal sensor disposed on the second substrate layer at a location near the periphery of the projection of the heater.

For example, the heater includes a central portion that has a first power density, and a peripheral portion surrounding the central portion that has a second power density higher than the first power density.

In yet other aspects, a zero-heat-flux DTT measurement device is constituted of a flexible substrate including a center section, a tab extending from the periphery of the center section, and a tail extending from the periphery of the center section. An electrical circuit disposed on a surface of the flexible substrate includes a heater trace defining a heater surrounding a zone of the surface, a first thermal sensor disposed in the zone, a second thermal sensor disposed on the tail, outside of the heater trace, and a third thermal sensor disposed on the tail, between the second thermal sensor and a peripheral portion of the heater trace. A plurality of electrical contact pads is disposed on the tab, and a plurality of conductive traces connect the first and second thermal sensors, a memory device, and the heater trace with the plurality of electrical contact pads.

For example, the heater has a central portion with a first power density and a peripheral portion surrounding the central portion with a second power density higher than the first power density.

### BRIEF DESCRIPTION OF THE DRAWINGS

FIG. 1 is a schematic block diagram of a first prior art deep tissue temperature measurement system including a ZHF temperature measurement device.

FIG. 2 is a schematic side sectional diagram of a second prior art deep tissue temperature measurement system including a ZHF temperature measurement device with an aluminum cap.

FIG. 3A is a plan view of an assembly including a substrate with a ZHF electrical circuit disposed on a surface of the substrate; and, FIG. 3B is a side sectional view of a ZHF temperature measurement device that incorporates the assembly of FIG. 3A.

FIG. 4A illustrates a plan view of an assembly including a substrate with a ZHF electrical circuit disposed on a surface of the substrate and FIG. 4B is a schematic diagram representing the ZHF electrical circuit of FIG. 4A.

FIG. 5 is an exploded view, in perspective, of a ZHF temperature measurement device incorporating the substrate assembly of FIG. 4A.

FIGS. 6A-6F illustrate steps to manufacture a ZHF temperature measurement device by incorporating the elements of FIGS. 4A and 5.

FIG. 7A is a first side sectional, partly schematic illustration of a zero-heat-flux DTT measurement device with a multi-layer construction.

FIG. 7B is a second side sectional, partly schematic illustration of the zero-heat-flux DTT measurement device of FIG. 7A rotated to illustrate additional elements of the multi-layer construction.

FIG. 8 is a block diagram illustrating a temperature measurement system.

FIG. 9 illustrates a second heater construction for the zero-heat-flux DTT measurement device of FIGS. 7A and 7B.

FIG. 10 is a flow chart illustrating a method of measuring body temperature using a zero-heat-flux temperature measurement device with peripheral skin temperature measurement.

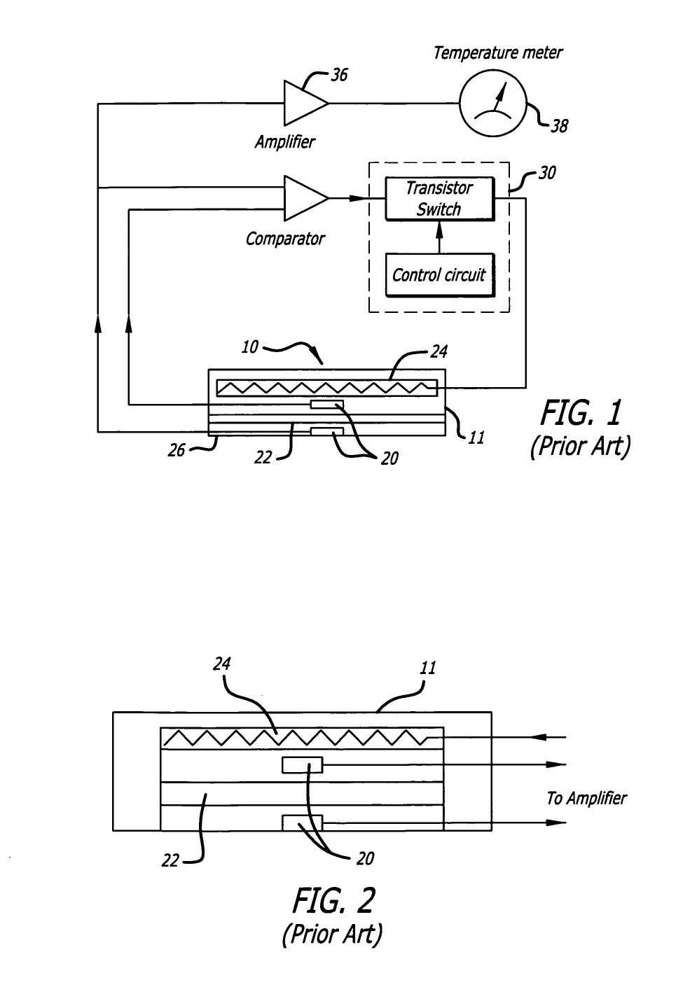

### DETAILED DESCRIPTION

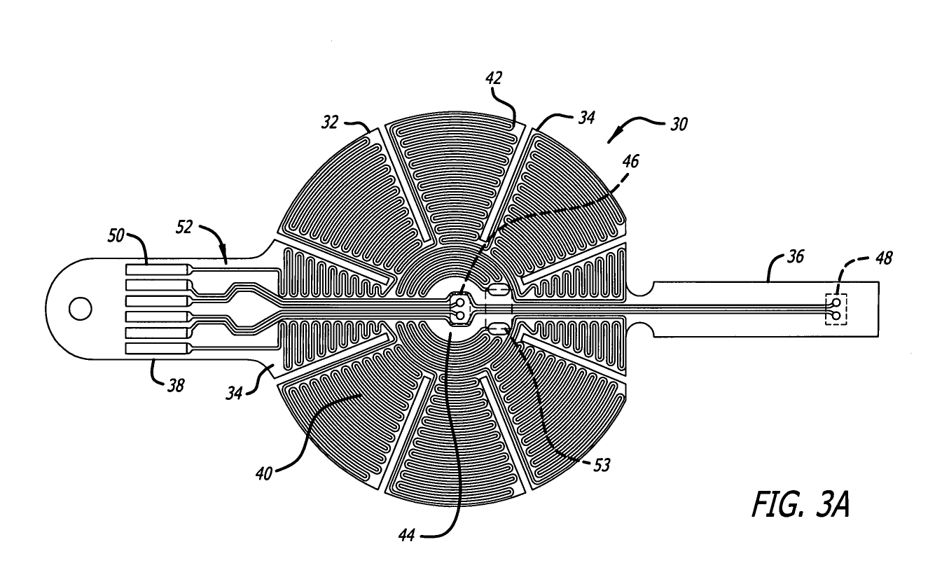

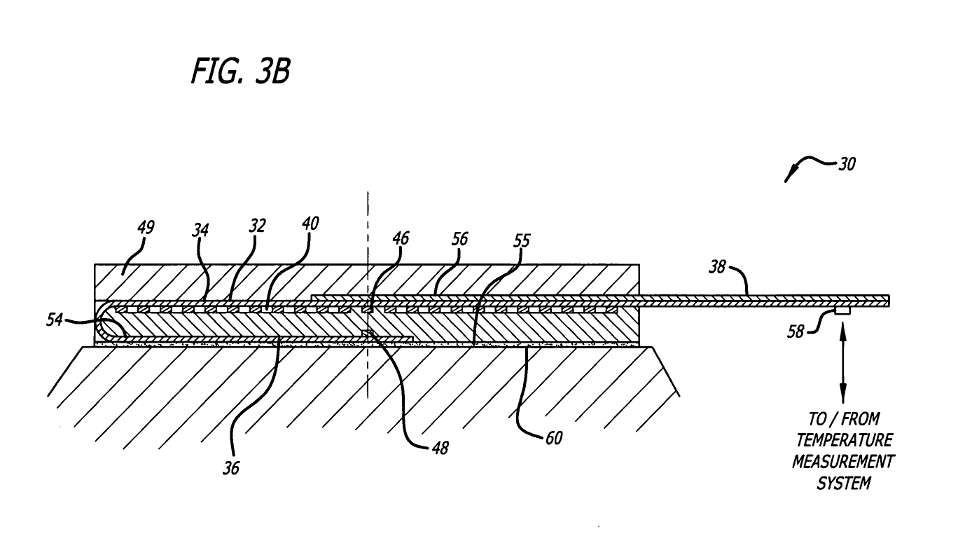

An inexpensive, disposable, zero-heat-flux DTT measurement device described and claimed in commonly-owned U.S. patent application Ser. No. 12/584,108 is illustrated in FIGS. 3A and 3B. The device is constituted of a flexible substrate 32 with central, tail, and tab sections 34, 36, and 38. A ZHF electrical circuit is disposed on a first side of the substrate. The electrical circuit includes a heater, thermal sensors, electrically-conductive traces, and mounting and contact pads. The heater 40 is defined by an electrically conductive heater trace 42 that surrounds an unheated zone 44 of the surface. A first thermal sensor 46 is disposed in the zone 44, and a second thermal sensor 48 is disposed outside of the heater trace on the tail section 36. Electrical contact pads 50 are disposed outside of the heater trace on the tab section 38, and a plurality of conductive traces 52 connects the thermal sensors and the heater trace with the plurality of contact pads. The continuity of the heater trace 42 is maintained by an electrically conductive zero-ohm jumper 53 which crosses, and is electrically isolated from, the two traces for the second thermal sensor 48. As per FIG. 3B, the ZHF temperature measurement device 30 is assembled by folding the central and tail sections 34 and 36 together to place the first and second thermal sensors 46 and 48 in vertical proximity to each other. A layer 54 of insulation disposed between the central and tail sections separates and provides a thermal resistance between the first and second thermal sensors 46 and 48. A flexible heater insulator 49 is attached to a second side of the substrate 32, over the central section 34. The device 30 is oriented for operation so as to position the heater 40 and the first thermal sensor 46 on one side of the layer of insulation 54 and the second thermal sensor 48 on the other side of the layer, and in close proximity to a skin surface area 60 where a measurement is to be taken. A layer 55 of adhesive on the lower side of the layer 54 attaches the device 30 to the skin surface area 60. The tab section 38 is stiffened by a flexible stiffener 56 disposed on a surface of the flexible substrate. The stiffener extends substantially coextensively with the tab section 38 and, preferably, at least partially over the center section 34. The layout of the electrical circuit on a single surface of the flexible substrate provides a low-profile ZHF temperature measurement device that is essentially planar. The device 30 includes a pluggable interface 58 to a temperature measurement system component of a patient vital signs monitoring system. In this regard, the tab 38 is stiffened and configured with the array of contact pads so as to be able to slide into and out of connection with the connector of an interface cable.

In the operation of a ZHF temperature measurement device such as is illustrated in FIG. 3B heat generated by the heater establishes and maintains an isothermal channel into tissue underneath the device. When the zero heat flux condition occurs, the temperature of the skin surface at the mouth of the isothermal channel is at a level substantially equal to the temperature of subsurface tissue at or near the deep tissue terminus of the isothermal channel. At this time, deep tissue temperature can be determined by measurement of the skin surface temperature using the thermal sensor closest to the skin. However, lateral heat dissipation in the skin can introduce error into the measurement.

Commonly-owned U.S. patent application Ser. No. 12/798,670 sets forth inexpensive, disposable, ZHF device constructions that utilize heaters in which power density increases in the direction of the heater's periphery. The rise in power density produces a uniform temperature from the center to the periphery of the heater that is intended to counter the effects of lateral heat dissipation in the skin by equalizing the skin temperature in the measurement area. It is desirable to provide these constructions with the ability to detect lateral heat dissipation in the skin, or to verify skin temperature equalization, by adding the ability to measure skin temperature at more than a single location.

In some aspects, a ZHF temperature measurement device is equipped with the ability to detect or monitor lateral heat dissipation in the skin surface area through which deep tissue temperature is to be measured. Detection of the condition enables more precise control of a heater constructed and operated to maintain a uniform temperature across the skin surface area where the measurement is made.

Consequently, it is desirable to provide a ZHF temperature measurement device with the capability of measuring a skin temperature difference in a lateral direction of the surface of the device which contacts a skin surface area where a DTT measurement is to be made. In some aspects, it is desirable to measure the temperature difference across a skin surface area that coincides with a surface of the heater. In still other aspects it is particularly desirable to measure a temperature difference from an inner portion to a peripheral portion of the skin surface area.

A temperature device for zero-heat-flux temperature measurement includes first and second flexible substrate layers sandwiching a layer of thermally insulating material, in which a heater trace disposed on the first substrate layer defines a heater facing one side of the layer of thermally insulating material. The heater includes a central portion surrounding a first thermal sensor and a peripheral portion surrounding the central portion. A second thermal sensor is disposed on the second substrate layer facing an opposing side of the layer of thermally insulating material, and a third thermal sensor is disposed on the second substrate layer facing the opposing side of the layer of thermally insulating material. The second and third thermal sensors are separated so that, when the device is in use, the second thermal sensor is located near a central portion of a skin surface area being measured and the third thermal sensor is located near a peripheral portion of the skin surface area.

In preferred constructions, the ZHF temperature measurement device includes a flexible circuit assembly including a flexible substrate supporting at least the heater, the thermal sensors, and the separating thermal insulator. In a preferred multilayer structure, the flexible substrate is folded about the thermal insulator so as to place the first and second layers adjacent opposing sides of the thermal insulator.

Although temperature device constructions are described in terms of preferred embodiments comprising representative elements, the embodiments are merely illustrative. It is possible that other embodiments will include more elements, or fewer, than described. It is also possible that some of the described elements will be deleted, and/or other elements that are not described will be added. Further, elements may be combined with other elements, and/or partitioned into additional elements.

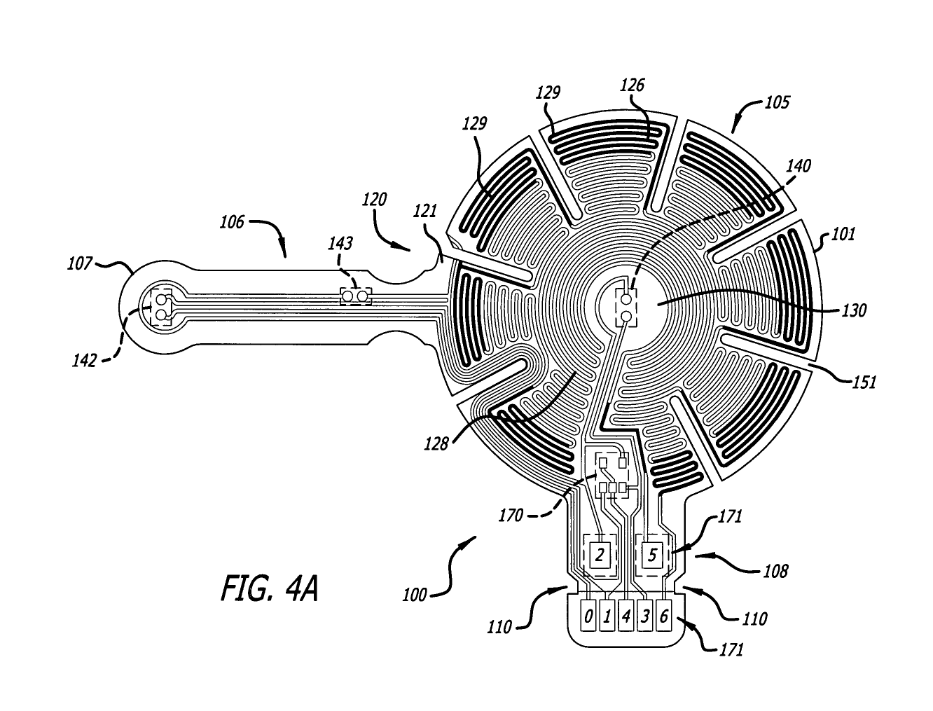

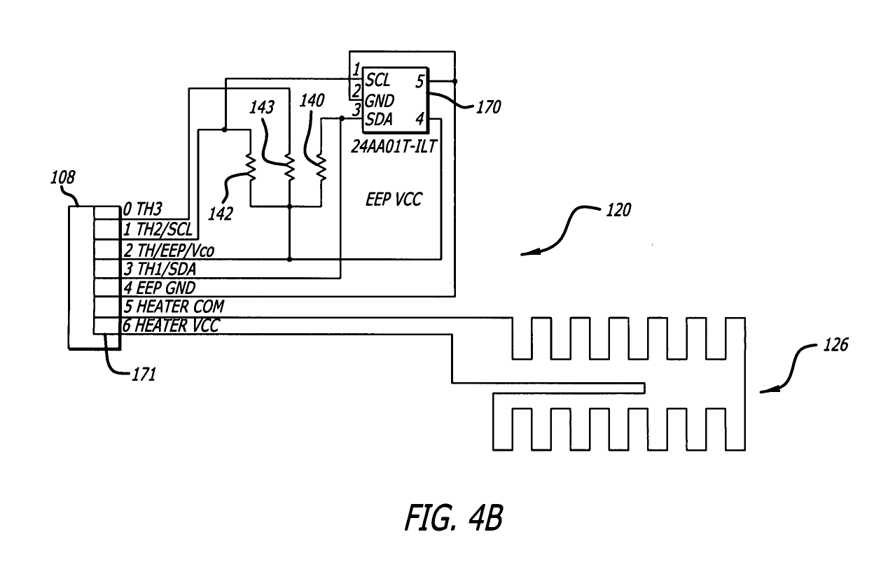

FIG. 4A illustrates a flexible circuit assembly used in a first construction of a zero-heat-flux temperature measurement device equipped with peripheral skin temperature measurement. The flexible circuit assembly 100 includes a flexible substrate 101. Preferably, but not necessarily, the flexible substrate 101 has contiguous sections 105, 106, and 108. Preferably, but not necessarily, the first, or center, section 105 is substantially circular in shape. The second section (or “tail”) 106 has the shape of a narrow, elongated rectangle with a bulbous end 107 that extends outwardly from the periphery of the center section 105 in a first direction. The third section (or “tab”) 108 is an extended section, preferably having the shape of a wide rectangle that extends outwardly from the periphery of the center section 105 in a second direction. Opposing notches 110 are formed in the tab 108 to receive and retain respective spring-loaded retainers of a connector. Preferably but not necessarily, the tail 106 is displaced from the tab 108 by an arcuate distance of less than 180° in either a clockwise or a counterclockwise direction. For example, the tail 106 and tab 108 are displaced by 90° in the assembly shown in FIG. 4A.

As per FIG. 4A, a ZHF electrical circuit 120 is disposed on the flexible substrate 101. Preferably, but not necessarily, the elements of the electrical circuit 120 are located on one surface 121 of the flexible substrate 101. The electrical circuit 120 includes at least an electrically conductive heater trace, thermal sensors, electrically conductive connective trace portions, and electrical contact pads. The heater trace 124 defines a generally annular heater 126 surrounding a zone 130 of the substrate 101 into which no portion of the heater trace 124 extends; in this regard, the zone 130 is not directly heated when the heater operates. The zone 130 occupies a generally circular portion of the surface 121. More completely, the zone 130 is a cylindrical section of the substrate 101 which includes the portion of the surface 121 seen in FIG. 4A, the counterpart portion of the opposing surface (not seen in this figure), and the solid portion therebetween. Preferably, but not necessarily, the zone 130 is centered in the center section 105 and is concentric with the heater 126. A first thermal sensor 140 is mounted on mounting pads formed in the zone 130. A second thermal sensor 142 is mounted on mounting pads disposed outside of the generally annular heater 126; preferably, these mounting pads are formed generally near the end of the tail 106, for example, in or near the center of the bulbous end 107 of the tail. A third thermal sensor 143 is mounted on mounting pads disposed outside of the generally annular heater 126; preferably, these mounting pads are formed in the tail section 106, generally between the mounting pads for the second thermal sensor 142 and the periphery of the heater 126. Electrical contact pads (“contact pads”) 171 are formed on the surface 121, in the tab 108.

In some constructions, the ZHF electrical circuit 120 includes a thermal sensor calibration circuit 170 with at least one multi-pin electronic circuit device mounted on the assembly 100. For example, with reference to FIG. 4A, the thermal sensor calibration circuit 170 can be constituted of an electrically-erasable programmable read/write memory (EEPROM) mounted on mounting pads formed on a portion of the surface 121 on the center section 105 near or adjacent the tab 108.

Per FIG. 4A, a plurality of conductive trace portions connects the first, second, and third thermal sensors 140, 142, and 143, and the heater trace 124 (and the calibration circuit 170, if included) with the plurality of the contact pads 171. In those constructions that include a thermal sensor calibration circuit, at least one contact pad 171 may be shared by the thermal sensor calibration circuit 170 and one of the heater 126, the first thermal sensor 140, the second thermal sensor 142, and the third thermal sensor 143.

As seen in FIG. 4A, preferably, but not necessarily, the center section 105 has formed therein a plurality of slits 151 to enhance the flexibility and conformability of the flexible substrate. The slits extend radially from the periphery toward the center of the center section 105 to define zones which move or flex independently of each other. The layout of the heater trace 124 is adapted to accommodate the slits. In this regard, the heater trace follows a zigzag or switchback pattern with legs that increase in length from the periphery of the zone 130 to the ends of the slits 151 and then, after a step decrease at those ends, generally increase in length again to the outer periphery of the heater 126 in the zones defined by the slits. As illustrated, the construction of the heater has a generally annular shape centered on the zone 130, although the annularity is interrupted by the slits. Alternatively, the annular shape can be viewed as including a peripheral annulus of wedge-shaped heater zones surrounding a generally continuous central annulus.

Preferably, the heater 126 has a non-uniform power density heater structure that can be understood with reference to FIG. 4A. In this construction, the heater 126 includes a central portion 128 (indicated by lightly drawn lines) having a first power density and a peripheral portion 129 (indicated by heavily drawn lines) which surrounds the central portion 128 and has a second power density higher than the first power density. The heater trace 124 is continuous and includes two ends, a first of which transitions to contact pad 5, and the second to contact pad 6. However, because of the slits, each of the central and peripheral portions 128 and 129 includes a plurality of sections arranged in a sequence, in which the sections of the central portion alternate with the sections of the peripheral portion. Nevertheless, the annular structure of the heater arrays the sections of the central portion 128 generally in a central annulus around the zone 130, and arrays the sections of the peripheral portion 129 around the central portion 128. When the heater 126 is operated, the central portion 128 produces a central annulus of heat at the first power density surrounding the zone 130 and the peripheral portion 129 produces a ring-shaped annulus of heat at the second power density that surrounds the central annulus of heat.

Preferably the heater trace 124 is continuous, but exhibits a nonuniform power density along its length such that the central heater portion 128 has a first power density and the peripheral portion 129 has a second power density that is greater than the first power density. With this configuration, a driving voltage applied to the heater 126 will cause the central heater portion 128 to produce less power per unit of heater area of the heater trace than the outer heater portion 129. The result will be a central annulus of heat at a first average power surrounded by a ring of heat a second average power higher than the first.

The differing power densities of the heater portions 128 and 129 may be invariant within each portion, or they may vary. Variation of power density may be step-wise or continuous. Power density is most simply and economically established by the width of the heater trace 124 and/or the pitch (distance) between the legs of a switchback pattern. For example, the resistance, and therefore the power generated by the heater trace, varies inversely with the width of the trace. For any resistance, the power generated by the heater trace also varies inversely with the pitch of (distance between) the switchback legs. Alternatively, the traces may have varying thicknesses at selected locations to vary the power density. For example, the central heater portion may have a heater trace with a thickness of x and the peripheral portion a thickness of 2x.

The electrical circuit 120 on the flexible substrate 101 seen in FIG. 4A is shown in schematic form in FIG. 4B. The contact pads 171 on the tab 108 numbered 0-6 in FIG. 4A correspond to the identically-numbered elements in FIG. 4B. The number of contact pads shown is merely for illustration. More, or fewer, contact pads can be used; any specific number is determined by design choices including the heater construction, the number of thermal sensors, the inclusion of a thermal sensor calibration circuit, and so on. In some constructions it is desirable to utilize one or more of the contact pads for electrical signal conduction to or from more than a single element of the electrical circuit 120 in order to minimize the number of contact pads, thereby simplifying the circuit layout, minimizing the size and mass of the tab 108, and reducing interface connector size.

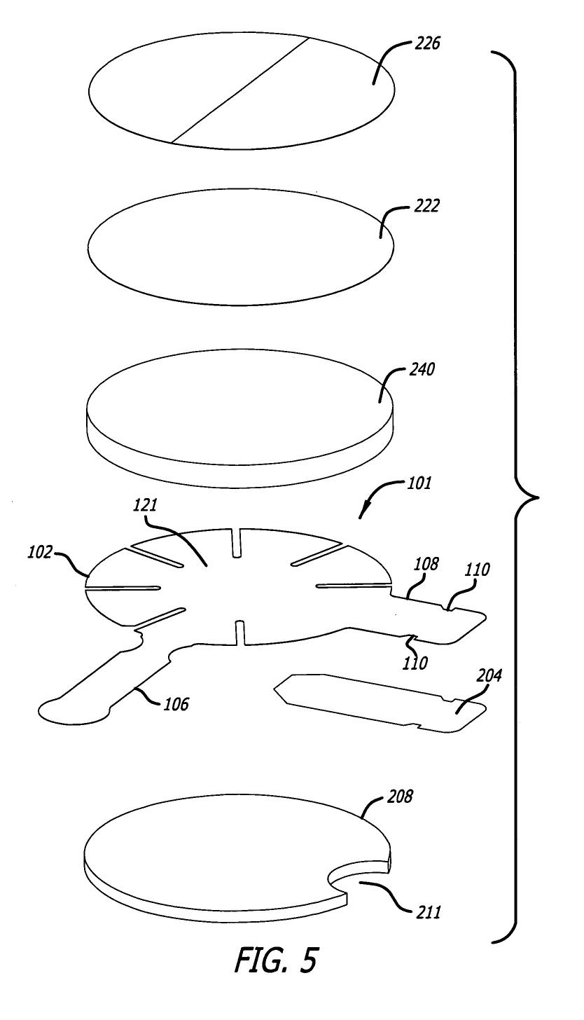

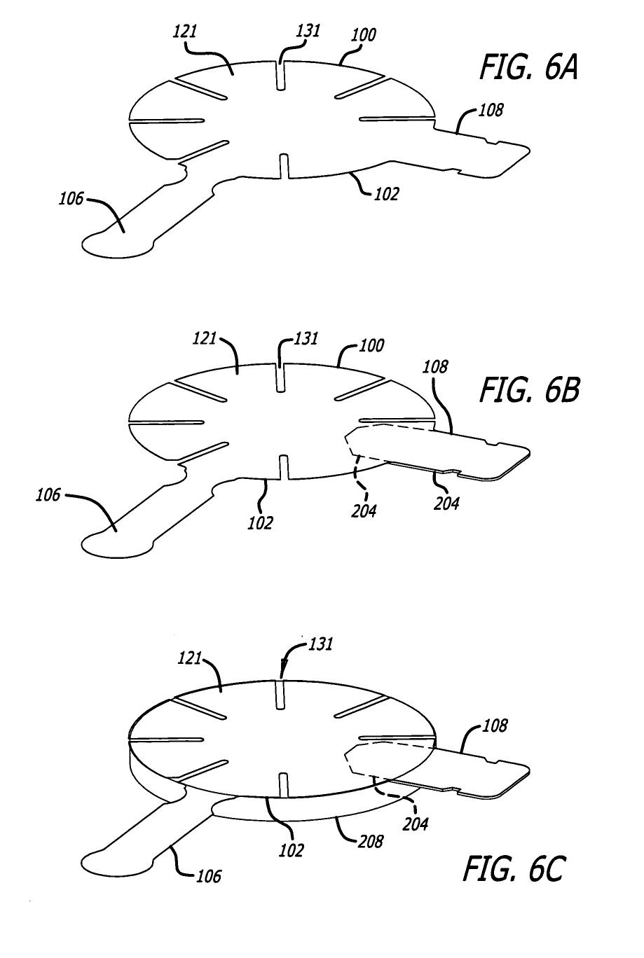

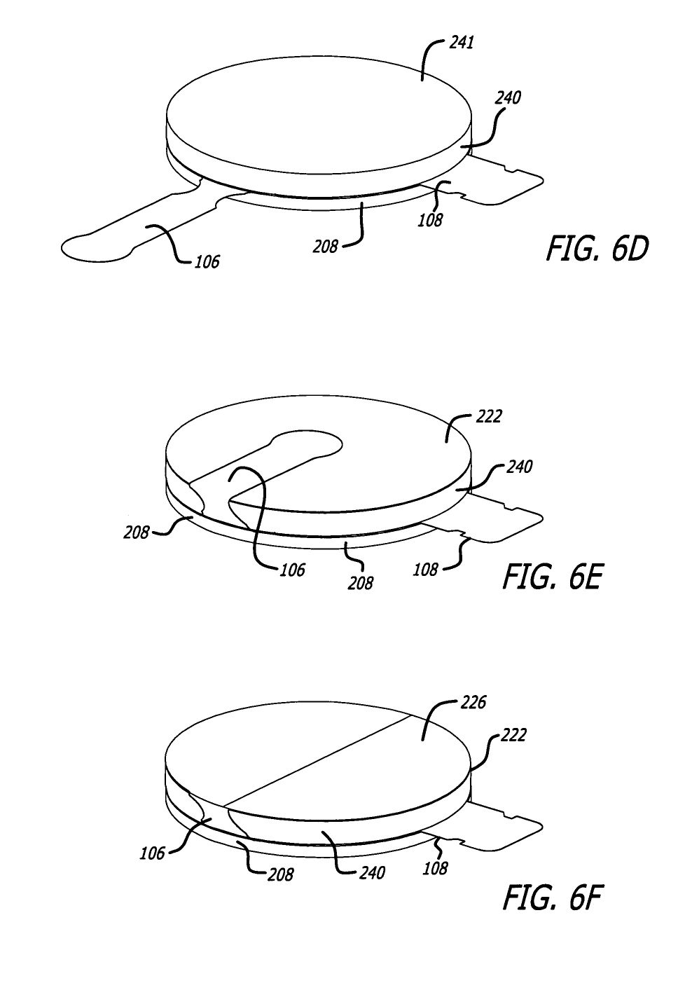

Fabrication of an electrical circuit on a flexible substrate greatly simplifies the construction of a disposable ZHF temperature measurement device, and substantially reduces the time and cost of manufacturing such a device. In this regard, manufacture of a ZHF temperature measurement device incorporating an electrical circuit laid out on a side of the flexible substrate 101 with the circuit elements illustrated in FIGS. 4A and 4B may be understood with reference to FIGS. 5 and 6A-6F. Although a manufacturing method is described in terms of specifically numbered steps, it is possible to vary the sequence of the steps while achieving the same result. For various reasons, some of the steps may include more operations, or fewer, than described. For the same or additional reasons, some of the described steps may be deleted, and/or other steps that are not described may be added. Further, steps may be combined with other steps, and/or partitioned into additional steps. Finally, in order to more clearly illustrate the assembly of the ZHF temperature measurement device, the details of the electrical circuit are not shown in FIGS. 5 and FIGS. 6A-6F.

Referring now to FIG. 5 and FIG. 6A, the traces, mounting pads, and contact pads for a ZHF electrical circuit are fabricated on the surface 121 (the “trace surface”) of a first side of the flexible substrate 101. The electronic elements (first, second, and third thermal sensors, and the calibration circuit 170, if included) are mounted to the mounting pads to complete an electrical circuit including the elements of FIG. 4A, laid out as shown in that figure. If used, the slits 131 separating the heater zones may be made in the center section 102 in this manufacturing step.

As per FIGS. 5 and 6B, in a second manufacturing step, a stiffener 204 is laminated to or formed on the surface (the “non-trace surface”) of a second side of the flexible substrate 101 within the area occupied by the tab 108. The stiffener has a portion shaped identically to the tab; when laminated to the second side, the stiffener extends over the tab and partially into the center section 102.

As per FIGS. 5 and 6C, in a third manufacturing step, a flexible layer 208 of insulating material is attached by adhesive or equivalent to the non-trace surface of the flexible substrate 101, over substantially the entire center section 102. This layer is provided to insulate the heater and the first thermal sensor from the ambient environment. As best seen in FIG. 5, this flexible layer may include a shaped recess 211 that receives a forward portion of a connector piece.

As per FIGS. 5 and 6D and in a fourth manufacturing step, a flexible layer 240 of insulating material is attached by adhesive or equivalent to the trace surface 121 of the flexible substrate, over substantially the entire center section 102. This layer covers the electrical circuit, except for the portions in the tail 106 and the tab 108.

As per FIGS. 5, 6D, and 6E, and in a fifth manufacturing step, a layer of adhesive 222 is applied over the surface 241 of the layer 240 and the tail 106 is folded over the layer 240 such that the first and second thermal sensors are maintained by the layer 240 in a preferred spaced relationship. The layer 240 of insulating material also separates and thermally isolates the second and third thermal sensors from the heater.

As per FIGS. 5 and 6F, in a sixth manufacturing step, a release liner 226 is attached to the layer of adhesive 222, over the central insulating layer with the tail folded thereto.

FIG. 6F illustrates an assembled ZHF temperature measurement device from an aspect showing the bottom of the device, that is, the surface area of the device that contacts the skin surface area where temperature is to be measured. When the device is used, the release layer 226 is stripped off to expose the layer of adhesive 222 by which the device is attached to the skin surface area.

A temperature measurement device according to this specification can be fabricated using the materials and parts listed in the following table. An electrical circuit with copper traces, mounting pads, and contact pads conforming to FIG. 4A can be formed on a flexible substrate of polyimide film by a conventional photo-etching technique and thermal sensors can be mounted using a conventional surface mount technique. The dimensions in the table are thicknesses, except that Ø signifies diameter. Of course, these materials and dimensions are only illustrative and in no way limit the scope of this specification or the claims which follow. For example, the heater and conductive traces may be made wholly or partly with electrically conductive ink.

Table of Materials and Parts

| | | Representative |
|---|---|---|
| | | dimensions/ |
| Element | Material/Part | characteristics |
| Flexible substrate | 2 mil thick Polyethylene | Substrate 101: |
| 101, heater 126, | terephthalate (PET) film | 0.05 mm thick |
| mounting pads, and | with deposited and | |
| contact pads | photo-etched ½ oz. | |
| | copper traces and pads | |
| | and immersion | |
| | silver-plated contacts. | |
| Thermal sensors | Negative Temperature | 10k thermistors in |
| 140, 142, 143 | Coefficient (NTC) | 0603 package. |
| | thermistors, Part # | |
| | R603-103F-3435-C, | |
| | Redfish Sensors. | |
| Flexible insulating | Closed cell polyethylene | Insulator 208: |
| layers 208, 240 | foam with skinned major | Ø40 mm × 3.0 mm thick |
| | surfaces coated with | Insulator 240: |
| | pressure sensitive | Ø40 mm × 3.0 mm thick |
| | adhesive (PSA) | |
| Stiffener 204 | 10 mil thick PET film | Stiffener: |
| | | 0.25 mm thick |
| Sensor calibration | Micron Technology | |
| circuit 770 | EEPROM, part # | |
| | 24AA01T-I/OT | |

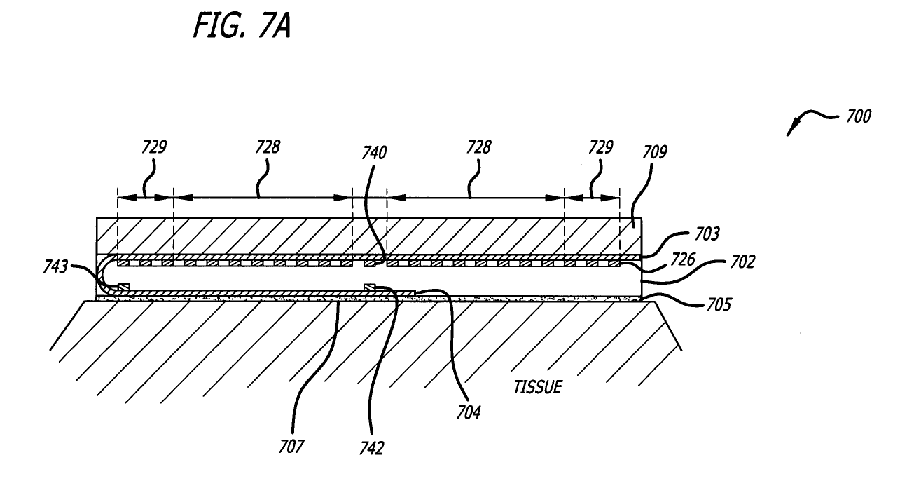

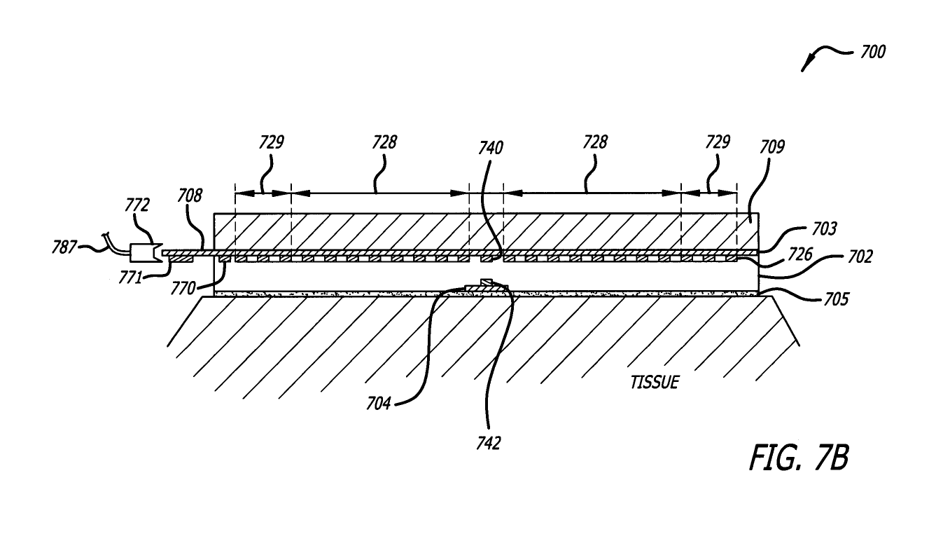

FIG. 7A is a sectional, partially-schematic illustration of a preferred zero-heat-flux temperature measurement device construction with peripheral skin temperature measurement. Preferably, but not necessarily the construction uses a flexible substrate assembly. As an example, the construction may use a flexible substrate with a ZHF electrical circuit such as the one shown in FIGS. 4A and 4B, assembled as shown in FIGS. 5 and 6A-6F. In this exemplary case, the view of FIG. 7A corresponds to a side section taken along the centerline of the tail folded as shown in FIG. 6E, and the view of FIG. 7B corresponds to a side section taken along the centerline of the tab when the device is assembled as shown in FIG. 6E. Not all elements of the measurement device are shown in these figures; however, the figures do show relationships between components of the construction that are relevant to zero-heat-flux measurement with peripheral skin temperature measurement.

As per FIG. 7A, the ZHF temperature measurement device 700 includes flexible substrate layers, a flexible layer of thermally insulating material, and an electrical circuit. The electrical circuit includes a heater 726, a first thermal sensor 740, a second thermal sensor 742, and a third thermal sensor 743. The heater 726 and the first thermal sensor 740 are disposed in or on a flexible substrate layer 703 and the second and third thermal sensors 742 and 743 are disposed in or on a flexible substrate layer 704. The first and second substrate layers 703 and 704 are separated and thermally isolated from one another by a flexible layer 702 of thermally insulating material. The flexible substrate layers 703 and 704 can be separate elements, but it is preferred that they be sections of a single flexible substrate folded around the layer of insulating material. Preferably, adhesive film (not shown) attaches the substrate to the insulating layer 702. A layer of adhesive material 705 mounted to one side of the substrate layer 704 is provided with a removable liner (not shown) to attach the measurement device to skin. Preferably, a flexible layer 709 of insulating material lies over the layers 702, 703, and 704 and is attached by adhesive film (not shown) to one side of the substrate layer 703; the layer 709 extends over the heater 726 and the first thermal sensor 740.

As seen in FIG. 7B, the electrical circuit includes a thermal sensor calibration circuit 770 and contact pads 771 disposed in or on the flexible substrate layer 703. The thermal sensor calibration circuit 770 is positioned outside of the heater 726, preferably between the heater 726 and the contact pads 771. The contact pads 771 are positioned on a section 708 of the substrate layer 703 that projects beyond the insulating layer 709 so as to be detachably coupled with a connector 772 fixed to the end of a temperature measurement system cable 787. Presuming that the thermal sensors 740, 742, and 743 are thermistors, the thermal sensor calibration circuit 770 includes a non-volatile semiconductor memory storing thermal sensor calibration information; such information can include one or more unique calibration coefficients for each thermal sensor. Although a stiffener is not shown in this figure, the section 708 may be stiffened in a manner corresponding to FIG. 3B by a flexible stiffener disposed on a surface of the flexible substrate between the substrate layer 703 and the layer 709.

With reference to FIG. 7A, when in use, the ZHF temperature measurement device 700 is disposed with the second and third thermal sensors 742 and 743 nearest the skin surface area through which a temperature measurement is to be taken. The layer 702 is sandwiched between the first and second substrate layers 703 and 704 so as to separate and thermally insulate the heater 726 and first thermal sensor 740 from the second and third thermal sensors 742 and 743. The device 700 includes a thin layer 705 of adhesive material to attach the device to a skin surface area where measurement is to take place. The second and third thermal sensors 742 and 743 are separated in a lateral direction of the surface 707 of the second layer 704 nearest the skin surface area when the device is in use. This lateral separation locates the second thermal sensor opposite the central portion 728 of the heater 726 and the third thermal sensor opposite the peripheral portion 729 of the heater 726. From another point of view, when the device is positioned on a skin surface area where temperature measurements are to be made, the lateral separation locates the second thermal sensor 742 near a central portion of the skin surface area and the third thermal sensor 743 near the periphery of the area.

When the device 700 is in use, the layer 702 acts as a large thermal resistance between the first thermal sensor 740 and the second and third thermal sensors 742 and 743. The second and third thermal sensors 742 and 743 sense skin temperatures in the skin surface area under the surface 707. Preferably, the second thermal sensor 742 senses a skin temperature in a central portion of the skin surface area, and the third thermal sensor 743 senses a skin temperature in a peripheral portion of a skin surface area. The first thermal sensor 740 senses the temperature of the top surface of the layer 702. In general, while the temperature sensed by the first thermal sensor 740 is less than the temperature sensed by the second thermal sensor 742, the heater is operated to reduce heat flow through the layer 702 and the skin. When the temperature of the layer 702 equals that of the thermal sensor 742, heat flow through the layer 702 stops and the heater is switched off. This is the zero-heat-flux condition as it is sensed by the first and second sensors 740 and 742. When the zero-heat-flux condition occurs, the temperature of the skin, indicated by the second thermal sensor, is interpreted as core body temperature. In some zero-heat-flux measurement device constructions, the heater 726 can include a central heater portion 728 that operates with a first power density, and a peripheral heater portion 729 surrounding the central heater portion that operates with a second power density higher than the first power density. Of course, the flexibility of the substrate permits the measurement device 700, including the heater 726, to conform to body contours where measurement is made.

Presume that the thermal sensor calibration circuit 770 includes a multi-pin electronically programmable memory (EEPROM) such as a 24AA01T-I/OT manufactured by Microchip Technology and mounted by mounting pads to the zero-heat-flux DTT measurement device 700. FIGS. 4A and 4B illustrate a construction in which one or more contact pads are shared by at least two elements of the electrical circuit.

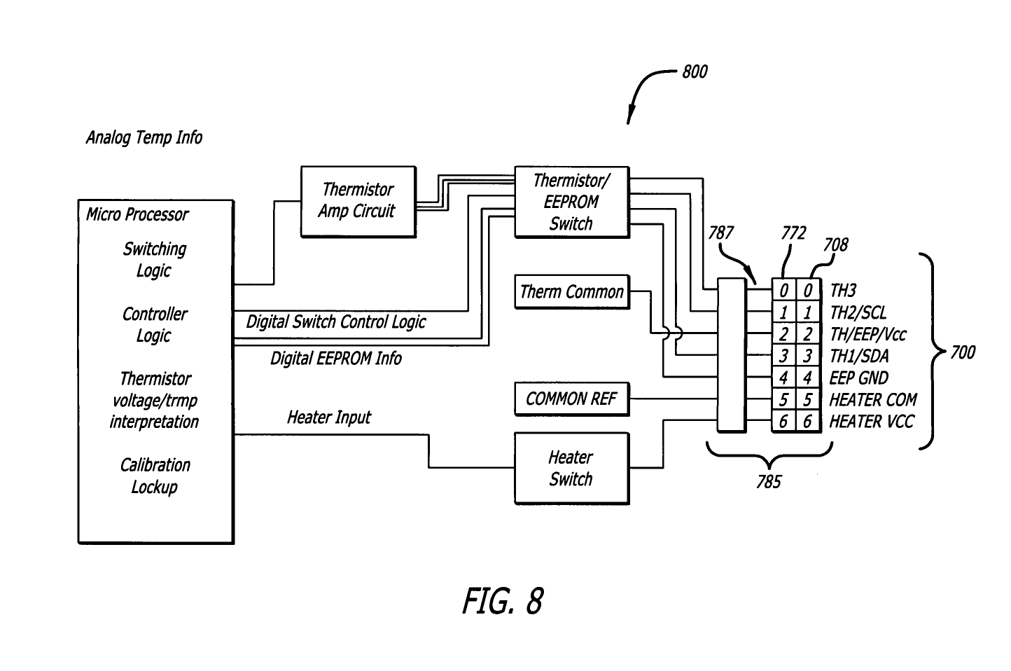

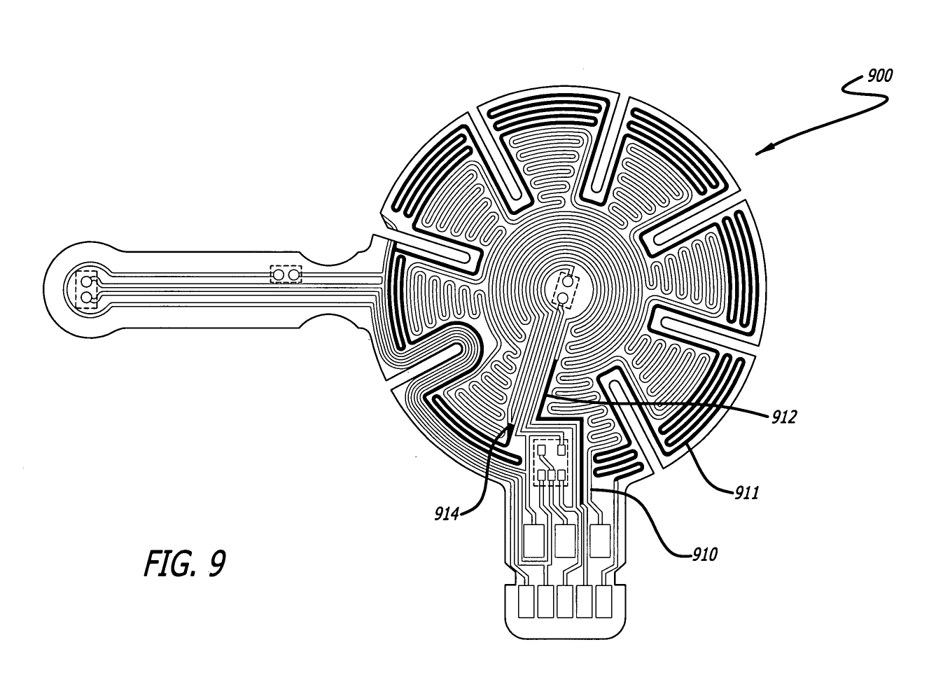

FIG. 8 illustrates a signal interface between a zero-heat-flux DTT measurement device according to FIGS. 7A and 7B, using the first flexible circuit construction of FIG. 4A as an example. With reference to these figures, a DTT measurement system includes control mechanization 800, a measurement device 700, and an interface 785 that transfers power, common, and data signals between the control mechanization and the measurement device. The interface can be wireless, with transceivers located to send and receive signals and a battery provided in the device 700 to power the electrical circuit. Preferably, the interface includes a cable 787 with a connector 772 releasably connected to the tab 708. The control mechanization 800 manages the provision of power and common signals on respective signal paths to the heater and provides for the separation of the signals that share a common signal path. A common reference voltage signal is provided on a single signal path to the thermal sensors, and respective separate return signal paths provide sensor data from the thermal sensors.

Presuming that the thermal sensor calibration circuit 770 includes an EEPROM, a separate signal path is provided for EEPROM ground, and the thermal sensor signal paths are shared with various pins of the EEPROM as per FIGS. 4A and 4B. This signal path configuration separates the digital ground for the EEPROM from the DC ground (common) for the heater in order to eliminate possibilities for damage to the EEPROM. In fact, it is desirable to electrically isolate the heater altogether from the other elements of the electrical circuit. Thus, as per FIG. 8, a first contact pad (contact pad 5, for example) of the plurality of contact pads is connected only to a first terminal end of the heater trace, while a second contact pad (contact pad 6, for example) of the plurality of contact pads is connected only to the second terminal end of the heater trace.

With reference to FIG. 4B, presume that the thermal sensors are negative temperature coefficient (NTC) thermistors. In this case, the common signal on contact pad 2 is held at a constant voltage level to provide Vcc for the EEPROM and a reference voltage for the thermistors. Control is switched via the thermistor/EEPROM switch circuit between reading the thermistors and clocking/reading/writing the EEPROM. Presuming again that the thermal sensors are NTC thermistors, the EEPROM has stored in it one or more calibration coefficients for each thermistor. When the device 700 is connected to the control mechanization, the calibration coefficients are read from the EEPROM through the SDA port in response to a clock signal provided to the SCL port of the EEPROM. The following Table of Signals and Electrical Characteristics summarizes an exemplary construction of the interface 785.

Table of Signals and Electrical Characteristics

| Element | Signals and Electrical Characteristics |
|---|---|
| Thermal sensors | Common reference signal is 3.3 volts DC. |
| 740, 742, 743 | Outputs are analog. |
| Heater 726 | Total resistance 4.5 to 7.0 ohms driven by a |
| | pulse width modulated waveform of 3.3 volts |
| | DC. The power density of the peripheral |
| | portion 729 is 30%-60% higher than that of |
| | the center portion 728. |
| Sensor calibration | Ground is 0 volts. Vcc is 3.3 volts DC. SCL |
| circuit 770 (Micron | and SDA pins see a low impedance source |
| Technology EEPROM | switched in parallel with the thermistor |
| 24AA01T-I/OT) | outputs. |

Calibration coefficients for the thermistors are obtained and stored in the EEPROM. The basis of obtaining accurate temperature sensing from the negative temperature coefficient thermistors is through calibration. In this regard, see U.S. patent application Ser. No. 12/798,668. During system operation, the control logic 800 determines the heater, central skin, and peripheral skin temperatures by applying calibration information to respective signals generated by the first, second, and third thermal sensors.

A second flexible substrate construction 900 with a useful for the measurement device 700 is illustrated in FIG. 9. According to the second construction, the electrical circuit corresponds to the electrical circuit 120 of FIGS. 4A and 4B, with the exception of the heater trace. In the second construction 900, the heater trace includes three traces: a first trace 910 that defines the central heater portion 728, a second trace 911, surrounding the first trace 910, that defines the peripheral heater portion 729, and a third trace 912 connected to the first and second traces at a shared node 914. The third trace 912 serves as a common connection between the first and second traces. This heater construction is thus constituted of independently-controlled central and peripheral heater portions that share a common lead. Alternatively, the construction can be considered as a heater with two heater elements. The power densities of the central and peripheral portions can be uniform or nonuniform. If the power densities of the two portions are uniform, the peripheral portion can be driven at a higher power level than the central portion so as to provide the desired higher power density. As per FIG. 9 the second heater construction utilizes three separate contact pads for the first, second, and third traces. Thus, for a construction of the electrical circuit that includes three thermal sensors and two independently-controlled heater portions that share a common lead, eight contact pads are provided on the tab.

In other constructions of the ZHF temperature measurement device 700, the flexible circuit assembly can be made with no slits, so that the heater 726 includes continuous central and peripheral portions 728 and 729 with different power densities. It is not necessary that the flexible substrate be configured with a circular central section, nor is it necessary that the annular heater be generally circular. In other constructions of the measurement device 700, the central substrate sections may have multilateral and oval (or elliptical) shapes, as may the heaters. All of the constructions previously described can be adapted to these shapes in order to accommodate design, operational, and/or manufacturing considerations. In all of these regards, see U.S. patent application Ser. No. 12/798,668.

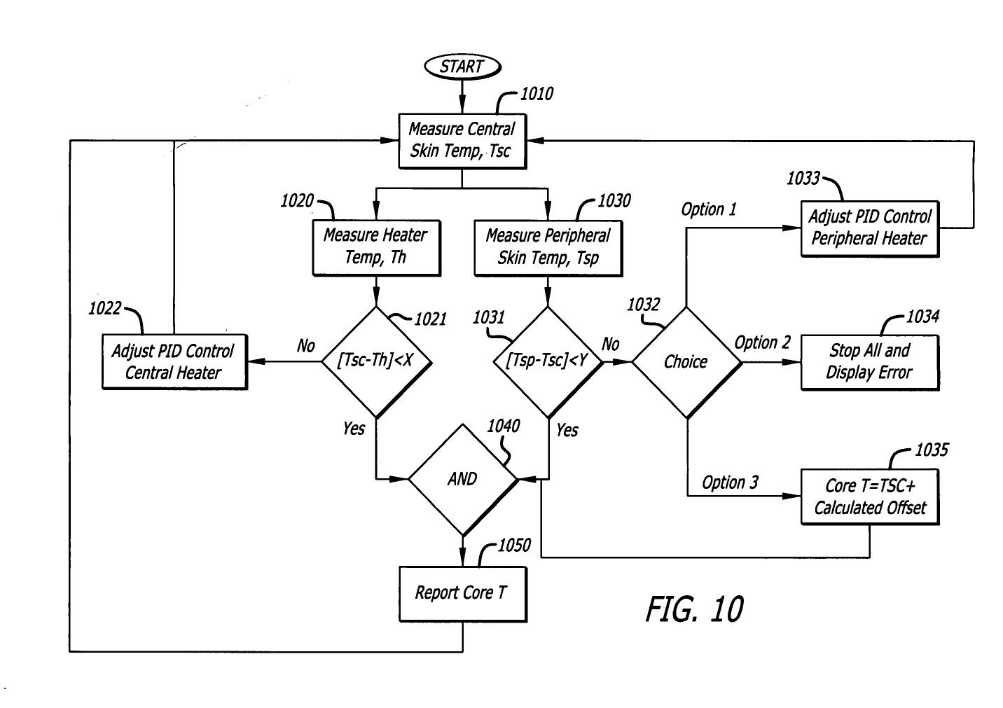

A method of temperature measurement using a zero-heat-flux temperature measurement device with peripheral skin temperature measurement is illustrated in FIG. 10. Presume that the device is deployed for use in the manner illustrated in FIGS. 7A and 7B and connected for operation by the control mechanization illustrated in FIG. 8, the heater is operating, and the three thermal sensors are operating. Initially, the skin temperature T_{sc} near the center of the skin surface area is measured at step 1010 using a resistance value provided by thermal sensor 742 and calibration coefficients for the thermal sensor, the heater temperature Tₕ is measured at step 1020 using a resistance value provided by thermal sensor 740 and calibration coefficients for the thermal sensor, and the skin temperature Tₛₚ near the periphery of the skin surface area is measured at step 1030 using a resistance value provided by thermal sensor 743 and calibration coefficients for the thermal sensor. Step 1021 checks the difference between the heater and central skin temperatures, and the loop 1010/1020/1021/1022 adjusts the heater output so as to maintain the difference within a range±X. Step 1031 checks the difference between the central and peripheral skin temperatures against a range±Y, and the loop 1010/1030/1031/1032 provides control options when the test is not satisfied. When the range conditions of 1021 and 1031 are concurrently satisfied as per the test in step 1040, the central skin temperature is reported as body core temperature.

The options out of step 1032 are representative of extra margins of ZHF temperature measurement system control provided by measurement of skin temperature at a peripheral margin of the skin surface area. In this regard, a heater operating with multiple power densities may be inadequate to maintain a substantially uniform temperature from the center to the periphery of the heater. For example, if the environment is very cold, peripheral heat loss through the skin may overcome the heater's ability to compensate. The third thermal sensor (143, 743) enables a mechanism and a method for evaluating a non-uniform thermal condition and initializing an option in response thereto. FIG. 10 illustrates three such options. First, if the central and peripheral heater portions are separately controllable, as per the heater layout in FIG. 9, the peripheral heater can be driven at 1033 to produce more heat in an effort to overcome the condition. Second, operation of the ZHF circuit can be suspended at 1034 and an error or alarm signal can be sounded and/or displayed. Third, the skin temperature T_{sc} can be adjusted by a calculated offset and the adjusted measurement submitted to the test at 1040. Other, or alternate, options may also be provided.

Although principles of temperature measurement device construction and manufacture have been described with reference to presently preferred embodiments, it should be understood that various modifications can be made without departing from the spirit of the described principles. Accordingly, the principles are limited only by the following claims.

1. A zero-heat-flux temperature device, comprising:
first and second flexible substrate layers sandwiching a layer of thermally insulating material;
a heater trace disposed on the first substrate layer defining a heater facing one side of the layer of thermally insulating material, the heater including a central portion surrounding a zone of the first substrate layer having no heater trace and a peripheral portion surrounding the central portion;
a first thermal sensor disposed in the zone;
a second thermal sensor disposed on the second substrate layer facing an opposing side of the layer of thermally insulating material;
a third thermal sensor disposed on the second substrate layer facing the opposing side of the layer of thermally insulating material; and,
the second and third thermal sensors separated so as to locate the second thermal sensor opposite a central portion of the heater and the third thermal sensor opposite the peripheral portion of the heater.

2. The zero-heat-flux temperature device of claim 1, in which the central portion of the heater has a first power density, the peripheral portion of the heater has a second power density, and the second power density is greater than the first power density.

3. The zero-heat-flux temperature device of claim 2, in which the heater trace includes a continuous heater trace having two ends, each of the central and peripheral portions includes a plurality of sections arranged in a sequence, and sections of the central portion alternate with sections of the peripheral portion.

4. The zero-heat-flux temperature device of claim 2, in which the central portion of the heater includes a first heater trace portion, the peripheral portion of the heater includes a second heater trace portion separate from the first heater trace portion, and the heater trace further includes a common heater trace portion and connected at a shared node to the first and second heater trace portions.

5. The zero-heat-flux temperature device of claim 1, further including a programmable memory storing thermal sensor calibration information.

6. The zero-heat-flux temperature device of claim 1, in which a flexible substrate has a construction that includes a center section, a tab extending outwardly from the periphery of the center section, and a tail extending outwardly from the periphery of the center section, a plurality of contact pads is disposed on the tab, a plurality of conductive traces connects the first, second, and third thermal sensors and the heater trace with the plurality of contact pads, and the center section and the tail are folded around the layer of thermal insulating material such that the center section constitutes the first substrate layer and the tail constitutes the second substrate layer.

7. The zero-heat-flux temperature device of claim 6, in which a programmable memory storing thermal sensor calibration information is disposed on the flexible substrate and conductive traces of the plurality of conductive traces connect the programmable memory with contact pads of the plurality of contact pads.

8. A temperature measurement device, comprising:
a flexible substrate including a first section, a tab section extending outwardly from a periphery of the first section, and a tail section extending outwardly from the periphery of the first section; and,
an electrical circuit on a surface of the flexible substrate, the electrical circuit including a heater trace on the first section defining a central heater portion surrounding a zone of the substrate with no heater trace and a peripheral heater portion surrounding the central heater portion, a first thermal sensor disposed in the zone, second and third thermal sensors disposed on the tail section, a plurality of contact pads disposed outside of the heater trace, and a plurality of conductive traces connecting the first, second, and third thermal sensors and the heater trace with the plurality of contact pads.

9. The temperature measurement device of claim 8, in which the central heater portion is a first power density portion, the peripheral heater portion is a second power density portion, and the second power density is greater than the first power density.

10. The temperature measurement device of claim 9, in which the heater trace includes a continuous heater trace having two ends, each of the central and peripheral heater portions includes a plurality of sections arranged in a sequence, and sections of the central heater portion alternate with sections of the peripheral heater portion.

11. The temperature measurement device of claim 9, in which the central heater portion includes a first heater trace portion, the peripheral heater portion includes a second heater trace portion separate from the first trace portion, and the heater trace further includes a common heater trace portion separate from the first and second heater trace portions and connected at a shared node to the first and second heater trace portions.

12. The temperature measurement device of claim 9, in which the electrical circuit includes a programmable memory storing thermal sensor calibration information and conductive traces of the plurality of conductive traces connect the programmable memory with contact pads of the plurality of contact pads.

13. The temperature measurement device of claim 8, in which the electrical circuit includes a programmable memory storing thermal sensor calibration information and conductive traces of the plurality of conductive traces connect the programmable memory with contact pads of the plurality of contact pads.

14. A temperature measuring system, comprising:
a zero-heat-flux temperature device with first and second flexible substrate layers sandwiching a layer of thermally insulating material, a heater trace disposed on the first substrate layer defining a heater facing one side of the layer of thermally insulating material, a first thermal sensor disposed on the first substrate layer, a second thermal sensor disposed on the second substrate layer facing an opposing side of the layer of thermally insulating material, and a third thermal sensor disposed on the second substrate layer facing the opposing side of the layer of thermally insulating material, in which the second and third thermal sensors are separated so as to locate the second thermal sensor near central portion of the heater and the third thermal sensor near the peripheral portion of the heater; and,
a controller for being coupled to the zero-heat-flux temperature device to determine a heater temperature sensed by the first thermal sensor, a central skin temperature sensed by the second thermal sensor, and a peripheral skin temperature sensed by the third thermal sensor, and operate the heater in response to the heater temperature, the central skin temperature, and the peripheral skin temperature.

15. The temperature measuring system of claim 14, in which the controller is coupled to the zero-heat-flux temperature device by one of a wireless link and a cable.

16. The temperature measuring system of claim 15, in which the zero-heat-flux temperature device includes a programmable memory storing thermal sensor calibration information and the controller determines the heater, central skin, and peripheral skin temperatures by applying calibration information to respective signals generated by the first, second, and third thermal sensors.

17. A method of measuring body core temperature using a zero-heat-flux temperature measurement device in contact with a skin surface area of a person, comprising:
determining a skin temperature T_{sc} near the center of the skin surface area using a thermal sensor positioned near the center;
determining a heater temperature Tₕ using a thermal sensor positioned near a heater positioned to block heat flux from the skin surface area;
determining a skin temperature Tₛₚ near the periphery of the skin surface area using a thermal sensor positioned near the periphery;
determining a first difference between the heater and central skin temperatures;
determining a second difference between the central and peripheral skin temperatures;
and,
if the first difference is within a range±X and the second difference is within a range±Y, reporting skin temperature T_{sc} as body core temperature.

18. The method of claim 17, further comprising:
if the first difference is not within a range±X and or the second difference is not within a range±Y,
adjusting the heat produced at a periphery of the heater.

19. The method of claim 17, further comprising:
if the first difference is not within a range±X and or the second difference is not within a range±Y,
issuing an alarm or an error signal.

20. The method of claim 17, further comprising:
if the first difference is not within a range±X and or the second difference is not within a range±Y,
adjusting the skin temperature T_{sc} by an offset value and reporting the offset skin temperature T_{sc} as body core temperature.

21. The method of claim 17, further comprising:
if the first difference is not within a range±X or the second difference is not within a range±Y, adjusting the heat produced by the heater.
